(19) United States
(12) Patent Application Publication
(10) Pub. No.: US 2004/0161760 A1
(43) Pub. Date: Aug. 19, 2004

(54) Method of molecular diagnosis of chronic myelogenous leukemia

(76) Inventor: Hiroyuki Mano, Tochigi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/468,406

(22) PCT Filed: Mar. 1, 2002
(86) PCT No.: PCT/JP02/01901

(30) Foreign Application Priority Data
Mar. 1, 2001 (JP) 2001-056438

(51) Int. Cl. C12Q 1/68; G01N 33/574

(52) U.S. Cl. 435/6; 435/7.23

(57) ABSTRACT

A method for detecting chronic myeloid leukemia is provided. The method comprises the step of measuring expression level of a gene selected from the group consisting of IFIT-2 gene, LAGE-1 gene, BAGE gene, DDB1 gene, ETS2 gene, PIASy gene, PIASx-α gene, PIASx-β gene, and DAPK3 gene in a test tissue or a test cell; or determining expression profile of a gene group containing at least one of said genes so as to thereby detect chronic myeloid leukemia.

### TECHNICAL FIELD

This invention relates to a method for detecting chronic myeloid leukemia; a method for evaluating a compound for its effectiveness in treating or preventing chronic myeloid leukemia; a therapeutic or prophylactic agent for chronic myeloid leukemia containing the compound obtained by the evaluation method; a therapeutic or prophylactic agent for chronic myeloid leukemia containing such compound; and a method for determining the risk of suffering from chronic myeloid leukemia.

### BACKGROUND ART

Chronic myeloid leukemia (CML) is a malignant disease caused by abnormal clonal growth of pluripotent hematopoietic stem cells [Kantarjian, H. M. et al.: Blood 82: 691(1993)]. Onset of this disease is believed to be caused by BCR-ABL fusion protein which is produced as a result of reciprocal translocation between chromosomes 9 and 22, and this BCR-ABL fusion protein is believed to act as an active tyrosine kinase to induce uncontrolled cell growth [Era, T. et al.: Proc. Natl. Acad. Sci. USA 97: 1737(2000)]. This uncontrolled cell growth results not only in the increase of undifferentiated blood cells but also in the differentiated blood cells from malignant clone, and as a consequence, the peripheral blood exhibits leukocytosis, thrombocytosis, and like.

The phase when the malignant clone has not yet lost its differentiation ability is called chronic phase (chronic phase: CP), and in this phase, the patients suffer from relatively mild clinical conditions. In most patients in their chronic phase, blood cell count of the peripheral blood can be controlled by administering interferon (IFN) α or hydroxyurea (HU) [Silver, R. T. et al.: Blood 94: 1517(1999)]. However, malignant blasts of many chronic myeloid leukemia patients lose their differentiation potency after several years, and as a result, the patients exhibits “acute leukemia” like conditions in which only the undifferentiated CML blasts propagate with the decrease in the mature blood cells. This phase is called blast crisis (blast crisis: BC) phase, and some patients are observed to experience accelerated phase (accelerated phase: AP) in the transition from the chronic phase to the blast crisis phase.

Once the patient is in the blast crisis phase, 50% survival period of the patient is generally as short as about 3 months, and the prognosis is extremely severe. Compared to the cells generally found in acute myeloid leukemia, CML blasts of the patient in the blast crisis phase are resistant to chemotherapy, and the prognosis is difficult to improve even by bone marrow transplantation. In order to improve the prognosis of the patients, it is therefore quite important to clarify the mechanism of the transition from the chronic phase to the accelerated phase or the blast crisis phase at the molecular level to thereby stop such transition. In contrast to the onset of the chronic myeloid leukemia defined by the production of the BCR-ABL fused gene product, the molecular mechanism that results in the advance into the accelerated phase or the blast crisis phase is little clarified. While activation of RAS oncogene product associated with point mutation [LeMaistre, A. et al.: Blood 73: 889(1989); Cogswell, P. C. et al.: Blood 74: 2629(1989)], and inactivation of p53 tumor suppressor gene product [Guran, S. et al.: Haematologia 29: 181(1998); Peller, S. et al.: Gene Chromosome Canc. 21: 2(1998)] have been reported to be associated with the advance of the disease phase, frequency of such events in the patient's specimen is not very high, and these events are unlikely to be the general mechanisms that are responsible for the advance of the chronic myeloid leukemia phase. While abnormality of chromosome 17 where p53 gene is located is often found in the late stage of chronic myeloid leukemia, Fioretos et al. describes that abnormality in the amino acid sequence of the p53 protein is rather rare in these cases, and Fioretos et al. postulates the presence of an unidentified tumor suppressor gene on the same chromosome whose gene abnormality plays an important role in the advance of the chronic myeloid leukemia phase [Fioretos, T. et al.: Blood 94: 225(1999)].

DNA microarray (also referred to as a DNA chip) is a recently developed system for analyzing gene expression, and this system has enabled macroscopic observation of gene expression of several thousands to tens of thousands of genes by repeating the experiment for several times. However, no report is so far known that has analyzed gene expression profile of the chronic myeloid leukemia patients by such system.

### DISCLOSURE OF THE INVENTION

An object of this invention is to provide a method for detecting chronic myeloid leukemia; a method for detecting risk factor for chronic myeloid leukemia; a method for evaluating a compound for its effectiveness in treating or preventing chronic myeloid leukemia; a compound having therapeutic or prophylactic effects obtained by such evaluation method; and a therapeutic or prophylactic agent for chronic myeloid leukemia containing such compound as its effective component.

The inventors of the present invention have made an extensive investigation to solve the problems as described above, and succeeded in identifying the genes whose expression level alters in phase-specific manner in the AC133 protein-positive hematopoietic stem cell of the patients suffering from chronic myeloid leukemia. The present invention has been completed on the bases of such identification.

This invention provides a method for detecting chronic myeloid leukemia comprising the step of measuring expression level of a gene selected from the group consisting of IFIT-2 gene, LAGE-1 gene, BAGE gene, DDB1 gene, ETS2 gene, PIASy gene, PIASx-α gene, PIASx-β gene, and DAPK3 gene in a test tissue or a test cell; or determining expression profile of a gene group containing at least one of said genes to thereby detect chronic myeloid leukemia. IFIT-2 gene is the gene for the interferon-induced protein represented by the nucleotide sequence shown in SEQ ID NO: 1 [Levy, Det al.: Proc. Natl. Acad. Sci. USA 83: 8929(1986)], or its allelic variant; LAGE-1 gene is the gene represented by the nucleotide sequence shown in SEQ ID NO: 3 [Lethe, B et al.: Int. J. Cancer 76: 903(1998)] or its allelic variant; BAGE gene is the gene represented by the nucleotide sequence shown in SEQ ID NO: 5 [Boel, P et al.: Immunity 2: 167(1995)] or its allelic variant; DDB1 gene is the gene for a DNA damage-binding protein represented by the nucleotide sequence shown in SEQ ID NO: 7 [Dualan, R et al.: Genomics 29: 62(1995)] or its allelic variant; ETS2 gene is the gene represented by the nucleotide sequence shown in SEQ ID NO: 9 [Watson, D. K. et al.: Proc. Natl. Acad. Sci. USA 85: 7862(1988)] or its allelic variant; PIASy gene is the gene represented by the nucleotide sequence shown in SEQ ID NO: 11 [Liu, B et al.: Proc. Natl. Acad. Sci. USA 95: 10626(1998)] or its allelic variant; PIASx-α gene is the gene represented by the nucleotide sequence shown in SEQ ID NO: 13 [Liu, B et al.: Proc. Natl. Acad. Sci. USA 95: 10626(1998)] or its allelic variant; PIASx-β gene is the gene represented by the nucleotide sequence shown in SEQ ID NO: 15 [Liu, B et al.: Proc. Natl. Acad. Sci. USA 95: 10626(1998)] or its allelic variant; and DAPK3 gene is the gene represented by the nucleotide sequence shown in SEQ ID NO: 17 [Kawai, T. et al.: Mol. Cell. Biol. 18: 1642(1998)] or its allelic variant. The genes as described above are summarized in Table 1, below.

TABLE 1

| | | SEQ ID NO: | SEQ ID NO: |
|---|---|---|---|
| Name of | Accession No. | (Nucleotide | (Amino acid |
| the gene | (GenBank) | sequence) | sequence) |
| IFIT-2 | M14660/X07557 | SEQ ID NO: 1 | SEQ ID NO: 2 |
| LAGE-1 | AJ223041 | SEQ ID NO: 3 | SEQ ID NO: 4 |
| BAGE | U19180 | SEQ ID NO: 5 | SEQ ID NO: 6 |
| DDB1 | U18299 | SEQ ID NO: 7 | SEQ ID NO: 8 |
| ETS-2 | J04102 | SEQ ID NO: 9 | SEQ ID NO: 10 |
| PIASy | AF077952 | SEQ ID NO: 11 | SEQ ID NO: 12 |
| PIASx-α | AF077953 | SEQ ID NO: 13 | SEQ ID NO: 14 |
| PIASx-β | AF077954 | SEQ ID NO: 15 | SEQ ID NO: 16 |
| DAPK3 | AB007144/NM001348 | SEQ ID NO: 17 | SEQ ID NO: 18 |

The test cell used may be a bone marrow cell or a blood cell, and in particular, an undifferentiated stem cell which propagates in the patient suffering from chronic myeloid leukemia, namely, a pluripotent hematopoietic stem cell (in particular, AC133 protein-positive hematopoietic stem cell). The measurement of the expression level of the gene or the determination of the expression profile of the gene group may be conducted at the level of either mRNA or protein. The measurement of the expression level of the gene or the determination of the expression profile of the gene group at the mRNA level may be conducted, for example, by gene amplification or by using a DNA microarray. The measurement of the expression level of the gene or the determination of the expression profile of the gene group at the protein level may be conducted, for example, by means of immunological method or by using a protein chip.

This invention also provides a method for determining phase of the chronic myeloid leukemia wherein the phase of the chronic myeloid leukemia is determined by using the detection method of the chronic myeloid leukemia according to any one of the detection methods as described above. The phase may be selected from chronic phase, accelerated phase, and blastic phase.

This invention also provides a method for evaluating a compound for its effectiveness in treating or preventing chronic myeloid leukemia, comprising the steps of:

(a) administering the compound to be evaluated to a test animal or a test cell, or bringing the compound in contact with a test animal or a test cell; and

(b) confirming whether the compound regulates expression level of a gene selected from the group consisting of IFIT-2 gene, LAGE-1 gene, BAGE gene, DDB1 gene, ETS2 gene, PIASy gene, PIASx-α gene, PIASx-β gene, and DAPK3 gene, or a gene which is functionally equivalent to such gene in test animal or the test cell.

This invention also provides a method for evaluating a compound for its effectiveness in treating or preventing chronic myeloid leukemia, comprising the steps of:

(a) bringing the compound to be evaluated in contact with a test animal or a test cell containing a fusion gene of an expression-regulatory region of a gene selected from the group consisting of IFIT-2 gene, LAGE-1 gene, BAGE gene, DDB1 gene, ETS2 gene, PIASy gene, PIASx-α gene, PIASx-β gene, and DAPK3 gene; and a reporter gene; and

(b) measuring the level of the reporter gene expressed in the test animal or the test cell to evaluate the compound.

This invention also provides a method for evaluating a compound for its effectiveness in treating or preventing chronic myeloid leukemia, comprising the steps of:

(a) bringing the compound to be evaluated in contact with a protein encoded by a gene selected from the group consisting of IFIT-2 gene, LAGE-1 gene, BAGE gene, DDB1 gene, ETS2 gene, PIASy gene, PIASx-α gene, PIASx-β gene, and DAPK3 gene; and

(b) measuring the activity of the protein to evaluate the compound.

This invention also provides method for evaluating a compound for its effectiveness in treating or preventing chronic myeloid leukemia, comprising the steps of:

(a) bringing the compound to be evaluated in contact with a STAT protein whose activity is inhibited by a protein coded by PIASy gene or PIASx-β gene; and

(b) measuring the activity of the STAT protein to evaluate the compound.

This invention also provides a therapeutic or prophylactic agent for chronic myeloid leukemia containing the compound obtained by the evaluation method of any one of the evaluation method as described above as its effective component.

This invention also provides a reagent for research purpose, or an agent for detection, treatment, or prevention of chronic myeloid leukemia comprising an antibody directed against a protein encoded by a gene selected from the group consisting of IFIT-2 gene, LAGE-1 gene, BAGE gene, DDB1 gene, ETS2 gene, PIASy gene, PIASx-α gene, PIASx-P gene, and DAPK3 gene, as an active ingredient.

This invention also provides a method for determining the risk of being susceptible for chronic myeloid leukemia comprising the step of detecting genetic polymorphism which causes abnormality in expression level of a gene selected from the group consisting of IFIT-2 gene, LAGE-1 gene, BAGE gene, DDB1 gene, ETS2 gene, PIASy gene, PIASx-α gene, PIASx-P gene, and DAPK3.

This invention also provides a method for determining the risk of being susceptible for chronic myeloid leukemia comprising the step of detecting genetic polymorphism which causes abnormality in biological activity of a protein encoded by a gene selected from the group consisting of IFIT-2 gene, LAGE-1 gene, BAGE gene, DDB1 gene, ETS2 gene, PIASy gene, PIASx-α gene, PIASx-β gene, and DAPK3 gene.

This invention also provides a therapeutic or prophylactic agent for chronic myeloid leukemia comprising an antisense nucleic acid which is capable of specifically hybridizing with the mRNA of a gene selected from the group consisting of IFIT-2 gene, LAGE-1 gene, BAGE gene, DDB1 gene, and ETS2 gene.

This invention also provides a therapeutic or prophylactic agent for chronic myeloid leukemia comprising a ribozyme capable of specifically cleaving the mRNA of a gene selected from the group consisting of IFIT-2 gene, LAGE-1 gene, BAGE gene, DDB1 gene, and ETS2 gene.

Next, the present invention is described in further detail.

This invention relates to a unique method for detecting chronic myeloid leukemia which is quite different from conventional methods used for detecting chronic myeloid leukemia, wherein expression level of a particular gene or expression profile of a particular gene group in the test tissue or the test cell is used as an index for the detection of the chronic myeloid leukemia. The term “expression level”, as used herein, refers to an absolute or relative amount of the transcription product of the particular gene (hereinafter also referred to as the “mRNA level”) or an absolute, or relative amount of the translation product of the particular gene (hereinafter also referred to as the “protein level”). The term “expression profile”, as used herein, refers to a patternized profile of expression levels of a plurality of genes including the above-mentioned particular gene.

1. Detection Method of Chronic Myeloid Leukemia According to the Present Invention

The present invention has revealed that IFIT-2 gene, LAGE-1 gene, BAGE gene, DDB1 gene, ETS2 gene, PIASy gene, PIASx-α gene, PIASx-β gene, and DAPK3 gene exhibit unique alteration of expression level in the AC133-positive hematopoietic stem cell of patients suffering from chronic myeloid leukemia. Accordingly, these genes are useful as a marker gene for chronic myeloid leukemia, and chronic myeloid leukemia can be detected by measuring the expression level of such gene or by determining the expression profile of the gene group including such gene in the tissue from the donor (used interchangeably herein with “test tissue”) or the cell from the donor (used interchangeably herein with “test cell”). The test cell used may be a bone marrow cells or a blood cell, and preferably a pluripotent hematopoietic stem cell, and most preferably an AC133-positive hematopoietic stem cell, an Flt3/Flk2 (GenBank XM007174)-positive hematopoietic stem cell, or a KDR/Flk (GenBank NM002253)-positive hematopoietic stem cell. While the test cell used in the Examples of the present invention is the AC133-positive hematopoietic stem cell from bone marrow, it is to be noted that the AC133-positive hematopoietic stem cell is also found in peripheral blood [Blood 95: 3106-3112 (2000)], and a blood cell can also be used in the detection method of the present invention.

Specifically, if the expression level of the gene as described above in the test tissue or the test cell is increased or decreased in comparison with the expression level in the chronic myeloid leukemia revealed by the present invention, the donor can be evaluated as suffering from the chronic myeloid leukemia. To be more specific, the genes as described above exhibit alteration of the expression level as described below in each phase of the chronic myeloid leukemia.

More specifically, the expression level of IFIT-2 gene increases in the patient of chronic phase chronic myeloid leukemia to the level of about 2 to 500 times higher than that of the normal donor while the expression level reduces again in the accelerated phase and the blast crisis phase. In the case of LAGE-1 gene and BAGE gene, the expression level in the normal donor is either zero or at a very low level if expressed, and the expression is found only in the chronic phase and the expression level again reduces in the accelerated phase and the blast crisis phase. Therefore, if the expression level of such gene in the hematopoietic stem cell of a donor is found to have increased to the level equivalent to the level as described above, the donor can be determined to be a patient of chronic myeloid leukemia and the phase of the disease can be evaluated to be the chronic phase.

In the hematopoietic stem cells of the patient suffering from chronic myeloid leukemia of the accelerated phase or the blast crisis phase, the expression level of DDBL gene can increase to the level of about 20 to 50 times higher than that of the normal donor or the patient of chronic phase chronic myeloid leukemia, and/or the expression level of ETS2 gene can increase to the level of about 2 to 10 times higher than that of the normal donor or the patient of chronic phase chronic myeloid leukemia. Therefore, if the expression level of such gene in the hematopoietic stem cell of a donor is found to have increased to the level equivalent to the level as described above, the donor can be determined to be a patient of chronic myeloid leukemia and the phase of the disease can be evaluated to be the accelerated phase or the blast crisis phase.

In the hematopoietic stem cells of the patient suffering from chronic myeloid leukemia of the accelerated phase or the blast crisis phase, the expression level of PIASy gene, PIASx-α and β genes, and DAPK3 gene can uniquely decrease to the level of about ½ to 1/50, respectively, compared to the normal donor or the patient of chronic phase chronic myeloid leukemia. Therefore, if the expression level of such gene in the hematopoietic stem cell of a donor is found to have decreased to the level equivalent to the level as described above, the donor can be determined to be a patient of chronic myeloid leukemia and the phase of the disease can be evaluated to be the accelerated phase or the blast crisis phase.

The mRNA expression level for the gene can be measured by gene amplification (for example, by PCR or by real time RT-PCR), by using a DNA microarray, or by dot blot hybridization, slot blot hybridization, northern hybridization, or the like. The protein expression level for the gene can be measured by using a protein chip, by an immunological process (for example, by ELISA), or the like.

In addition, whether the donor suffers from chronic myeloid leukemia can be determined more accurately by specifying expression profile of a gene group including any one of the chronic myeloid leukemia marker genes for the biological sample from the donor, and comparing the resulting expression profile with the expression profile of the case which has been preliminarily identified as the expression profile of the chronic myeloid leukemia. In other words, by this means, even the unchanged expression level shown for a gene can also be monitored and utilized as a control in order to improve the accuracy and reliability of the measurement result. The expression profile is preferably determined by using a DNA microarray in view of convenience, sensitivity, reproducibility, and the like.

Furthermore, a DNA containing at least a part of the IFIT-2 gene, LAGE-1 gene, BAGE gene, DDB1 gene, ETS2 gene, PIASy gene, PIASx-β gene, PIASx-α gene, or DAPK3 gene can be used as a primer or a probe to analyze the gene or the mRNA of the gene. The term “at least a part”, as used herein, means that the oligonucleotide which is used for the primer or the probe comprises a nucleotide sequence containing at least 8 nucleotides corresponding to the DNA sequence of the present invention, and preferably a nucleotide sequence of at least 10 nucleotides, and more preferably a polynucleotide containing at least about 15 to 25 nucleotides having the corresponding sequence.

2. Evaluation of the Compound Which May be Effective in Treating or Preventing Chronic Myeloid Leukemia

A substance, which is capable of normalizing the expression level or the activity level of the chronic myeloid leukemia-marker gene product thereby to enable differentiation of the leukemia cell into a normal cell or suppression of the abnormal growth of the leukemia cell, is expected to be found among some of the compounds which can regulate the expression level of a chronic myeloid leukemia marker gene, the activity of the chronic myeloid leukemia-marker gene product, or the STAT protein activity inhibited by the chronic myeloid leukemia-marker gene. On the bases of such expectation, compounds which may be effective in treating or preventing chronic myeloid leukemia may be determined by the procedure as described below. The term “evaluation”, as used herein, is to a wide concept including both the screening and the validation of a compound.

(1) Evaluation Using Expression Level of Chronic Myeloid Leukemia Marker Gene for the Index

A test compound can be evaluated for its effectiveness in the treatment or prevention of the chronic myeloid leukemia by administering the test compound to or by bringing the test compound in contact with a test animal (for example, mouse, rat, or rabbit) or a test cell (for example, a human cell line of chronic myeloid leukemia, human blood cell, or AC 133-positive hematopoietic stem cell), and confirming whether the test compound regulates the expression level of IFIT-2 gene, LAGE-1 gene, BAGE gene, DDB1 gene, ETS2 gene, PIASy gene, PIASx-α gene, PIASx-β gene, DAPK3 gene, or a gene which is functionally equivalent to these genes (i.e. whether the test compound brings the expression level of such a gene to the normal level) in the test animal or the test cell. The test animal or the test cell used may also be an animal or a cell having artificially introduced therein a fusion gene of the region regulating the expression of the IFIT-2 gene, LAGE-1 gene, BAGE gene, DDB1 gene, ETS2 gene, PIASy gene, PIASx-α gene, PIASx-β gene, or DAPK3 gene with a reporter gene. The reporter gene used is not limited to any specific gene, and exemplary reporter genes include β-galactosidase gene, luciferase gene, green fluorescence protein gene, and the like.

(2) Evaluation Using Activity-Regulating Ability of the Chronic Myeloid Leukemia Marker Gene for the Index

A test compound can be evaluated for its effectiveness in the treatment or prevention of the chronic myeloid leukemia by bringing the test compound in contact with a protein coded by IFIT-2 gene, LAGE-I gene, BAGE gene, DDB1 gene, ETS2 gene, PIASy gene, PIASx-a gene, PIASx-β gene, or DAPK3 gene, and confirming whether the test compound regulates the activity of the protein.

(3) Evaluation Using the Ability of Regulating the Activity of STAT Protein for the Index

A test compound can be evaluated for its effectiveness on the treatment or prevention of the chronic myeloid leukemia by bringing the test compound in contact with a STAT protein whose activity is inhibited by the protein coded by PIASy gene, PIASx-α gene, or PIASx-β gene, and confirming that the test compound regulates the activity of the STAT protein.

3. Applications of the Antibody Against the Product of a Chronic Myeloid Leukemia Marker Gene

An antibody against the product of the chronic myeloid leukemia-marker gene can be produced by the procedure as described below. The term “antibody”, as used herein, refers to the entire molecule of the antibody which binds to the product of a chronic myeloid leukemia-marker gene which is the antigen, as well as its fragment (for example, Fab or F(ab′)₂ fragment). The antibody against the product of the chronic myeloid leukemia-marker gene can be produced by any of the techniques known in the art. For example, a monoclonal antibody can be isolated from the culture supernatant of the hybridoma or from the ascites of the mouse administered with the hybridoma, after preparing the cell fusion of a commercially available mouse myeloma cell with the splenocyte from the mouse immunized against the chronic myeloid leukemia-marker gene product. The monoclonal antibody can also be produced from an in vitro system using phage display method. The product of the chronic myeloid leukemia-marker gene used for the antigen does not necessarily have the entire amino acid structure, and may be a peptide having a partial amino acid structure, its variant, derivative, or fusion peptide with other peptides, and the product of the marker gene may be the one produced by any one of genetic engineering technique, biological process, and chemical synthesis. The antibody can be used as a research-purpose reagent used in identifying or quantitating the product of the chronic myeloid leukemia-marker gene in a human biological sample, or as an effective component in a diagnostic, therapeutic, or prophylactic agent for chronic myeloid leukemia. The antibody used in the present invention can also be used as a fragment such as Fab, F(ab′)₂, or its derivative, or as a chimeric antibody or a humanized antibody, all of which are included within the present invention. The product of the chronic myeloid leukemia-marker gene can be immunologically measured by any of the methods known in the art including fluorescein antibody technique, passive hemagglutination, and enzyme antibody technique.

4. Application of the Antisense Nucleic Acids, Which Can Hybridize with the mRNAs of Chronic Myeloid Leukemia Marker Gene, and the Ribozymes Capable of Cleaving Such mRNAs

Of the chronic myeloid leukemia marker genes revealed in this invention, IFIT-2 gene, LAGE-1 gene, BAGE gene, DDB1 gene, and ETS2 gene are found to exhibit increased mRNA expression levels in the AC133-positive hematopoietic stem cells derived from chronic myeloid leukemia patients. In particular, the expression levels for the DDB1 gene and the ETS2 gene are found to increase in the transition from the accelerated phase to the blast crisis phase in proportion to the malignancy of the chronic myeloid leukemia. Therefore, an antisense nucleic acid (for example, DNA or RNA) which suppresses expression of these genes by specifically hybridizing with the mRNA of these genes, a gene construct expressing such antisense nucleic acid, a ribozyme which specifically cleaves such mRNA, and a gene construct expressing such ribozyme are candidates for the therapeutic agent of the chronic myeloid leukemia. The antisense nucleic acid, the ribozyme, and the gene constructs expressing such antisense nucleic acid or ribozyme can be produced by a method known in the art by using the nucleotide sequence of the IFIT-2 gene, LAGE-1 gene, BAGE gene, DDB1 gene, or ETS2 gene.

5. Therapeutic or Prophylactic Agent for Chronic Myeloid Leukemia

Chronic myeloid leukemia marker genes are genes whose expression level markedly alters in the blood cell of the patient suffering from chronic myeloid leukemia. Therefore, a substance capable of regulating the expression level of the chronic myeloid leukemia marker gene so as to get it back to its normal level in the blood cell is useful for treating or preventing the chronic myeloid leukemia. Exemplary substances which regulate the expression level of the chronic myeloid leukemia marker gene in the blood cell back to the normal level include the compound evaluated in the above 2, the antibody obtained in the above 3, and the antisense nucleic acid, the ribozyme, and the gene constructs expressing these obtained in the above 4.

When such substance is used as a therapeutic or prophylactic agent for chronic myeloid leukemia, the substance can be formulated into an appropriate dosage form by mixing the substance with a carrier generally used in the art depending on the administration route. For example, in the case of oral administration, the substance may be formulated into a tablet, capsule, granule, powder, liquid, or other dosage form, and a solid formulation for oral administration may be prepared by using an excipient, a binder, or a lubricant, or in further combination with a colorant or a disintegrant. Exemplary excipients include lactose, starch, talc, magnesium stearate, crystalline cellulose, methyl cellulose, carboxymethylcellulose, glycerin, sodium alginate, and gum arabic. Exemplary binders include polyvinyl alcohol, polyvinylether, ethyl cellulose, gum arabic, shellac, and saccharose. Exemplary lubricants include magnesium stearate and talc. The colorant, disintegrant, and the like used may be those known in the art. The tablet may be coated by the method well known in the art. The liquid formulation may be an aqueous or oleaginous suspension, a solution, a syrup, a elixir, or the like which is prepared by the method generally used in the art. When an injection is prepared, the substance as described above may be mixed with a pH adjusting agent, a buffer, a stabilizer, an isotonic agent, a local anesthetic, or the like, and a subcutaneous, intramuscular, or intravenous injection may be produced by a conventional method. Exemplary bases used in producing a suppository include cacao butter, polyethylene glycol, and other oil base.

The dose of the resulting preparation can not be determined universally since the dose differs by the conditions, weight, age, and the like of the patient. However, the compound of the present invention is generally administered at a dose in the range of about 0.1 to 1000 mg, which is administered in 1 to 4 times a day. If desired, the administration may be accomplished at an interval of 1 to several days, or at an even longer interval.

6. Determination of Susceptibility for Chronic Myeloid Leukemia by Utilizing Genetic Polymorphism

Liability to suffer from chronic myeloid leukemia (susceptibility for chronic myeloid leukemia) can be determined by detecting the genetic polymorphism which results in the abnormal expression of the marker gene for chronic myeloid revealed by the present invention, or by the polymorphism which results in the abnormal activity of the product of such gene. Exemplary genetic polymorphisms include minisatellite DNA, microsatellite DNA, and SNP (single nucleotide polymorphism), and such genetic polymorphism may be identified as described below. Namely, genetic polymorphism may be identified by sequencing the region of the chronic myeloid leukemia marker gene revealed by the present invention and the region which may be associated with the increase or decrease of the expression level of such gene in both the group of normal donors and the group of chronic rheumatism patients to thereby determine the polymorphic site, and then calculating the allele frequency of the polymorphic site thus determined and finding the allele whose frequency is significantly higher in the patient group to thereby identify the polymorphism correlated to the chronic myeloid leukemia.

The genetic polymorphism thus determined is preferably detected, for example, by determining the nucleotide sequence of the polymorphic site of the genomic DNA from the donor; by using a method which utilizes the difference in physicochemical nature of the DNA sample or the difference of the restriction site which depends on the changed nucleotide sequence for the polymorphic site (for example, by the method utilizing the difference in the mobility of the DNA sample including the polymorphic site in gel electrophoresis, capillary electrophoresis, or other electrophoretic means); by using a detection probe which is appropriate for detecting the particular polymorphic site; or by a method using mass spectroscopy. The method used is not limited to those described above as long as the method is capable of detecting the genetic polymorphism as described above, and any method known in the art can be employed for the detection. In any of the detection method as described above, it is to be noted that the DNA sample to be assayed is a sample of human origin, and the type of the sample is not limited as long as the sample contains the DNA to be assayed. Exemplary samples are body fluids such as blood, bone marrow, semen, ascites, and urine; cells from the tissue such as liver; hairs such as those on the head. Genomic DNA can be extracted, purified, and prepared from these samples by the method generally used in the art.

In the method for detecting chronic myeloid leukemia risk factor according to the present invention, the donor is determined to have a high risk of suffering from chronic myeloid leukemia when the polymorphism detected for the donor is the one whose frequency is significantly high in a chronic myeloid leukemia patient, and the donor is determined to have a low risk of suffering from chronic myeloid leukemia when the polymorphism detected is not such type.

### BRIEF DESCRIPTION OF DRAWINGS

FIG. 1 is a schematic view showing pMX-tetOFF/PIASy-F constructed in Example 5.

FIG. 2A is a profile view showing cell number of KCL22 cell carrying pMX-tetOFF/PIASy-F or pMX-tetOFF introduced therein in relation to day of growth, and FIG. 2B is a profile view showing survival rate of KCL22 cell carrying pMX-tetOFF/PIASy-F or pMX-tetOFF introduced therein in relation to day of growth.

FIG. 3 is a micrograph of KCL22 cell carrying pMX-tetOFF/PIASy-F or pMX-tetOFF introduced therein.

### BEST MODE FOR CARRYING OUT THE INVENTION

Next, the present invention is described in further detail by referring to Examples of the invention which by no means limit the scope of the present invention.

### EXAMPLE 1

### Preparation of AC133 Positive Cell

AC133-positive hematopoietic stem cells were prepared from donors by the procedure as described below. First, bone marrow aspirates were obtained from 14 donors (normal donors: 2, chronic phase patient: 7, accelerated phase patient: 2, and blast crisis phase patient: 3) after obtaining informed consent. Next, bone marrow mononuclear cells (MNC) were isolated and purified from the thus obtained bone marrow aspirates by Ficoll-Hypaque density gradient centrifugation, and the cells were washed with PBS. The bone marrow mononuclear cells were then suspended in 300 μl MACS buffer [PBS/3% fetal calf serum (FCS)/2 mM EDTA], and AC133 positive cell in the bone marrow mononuclear cells were labeled with anti-AC133 MicroBeads by adding 100 μl each of FcR blocking reagent and solution of anti-AC133 antibody conjugated to magnetic beads included in the ACC133-positive cell isolation kit (Militenyi Biotec, Auburn, Calif.), and allowing the reaction to take place at 4° C. for 30 minutes to thereby label the AC133 positive cell in the bone marrow mononuclear cell with the anti-AC133 MicroBeads. After thoroughly washing the bone marrow mononuclear cells with MACS buffer, miniMACS column (Militenyi Biotec, Auburn, Calif.) was used to obtain the AC133 positive cells. When the resulting AC133 positive cells were evaluated for their purity by staining with Wright-Giemsa solution (Muto Pure Chemicals), juvenile hematopoietic stem cells constituted 80% or more of the total cells in the samples from all donors.

### EXAMPLE 2

### Preparation of cRNA

AC133 positive cells from each donor obtained in Example 1 were used to prepare cRNA for gene expression analysis. First, total RNA was purified from each AC133 positive cell by using RNAzol B reagent (TEL-TEST, Friendswood, Tex.). Next, to the resulting total RNA was added T7-dT primer: 5′-tctagtcgacggccagtgaattgtaatacgactcactatagggcgtttttttttttttttttt-3′ (SEQ ID NO: 19) to hybridize the primer to the mRNA in the total RNA, and cDNA chain complementary to the mRNA was synthesized by using reverse transcriptase SuperScript II (Life Technologies, Rockville, Md.). Using SuperScript choice system (Life Technologies, Rockville, Md.), a double-strand cDNA was then synthesized by using the resulting single strand cDNA as a template. cRNA was then synthesized from the resulting double-strand cDNA by using Ampliscribe transcription kit (Epicentre Technologies, Madison, MI). This procedure was repeated twice to finally obtain 5 to 70 μg of cRNA from the AC 133 positive cells of each patient.

### EXAMPLE 3

### Analysis of Gene Expression Using DNA Microarray

ExpressChip DNA microarray system (Mergen, San Leandro, Calif.) and the cRNA obtained in Example 2 were used to analyze the gene expression in the AC133 positive cell from each donor. Double-stranded cDNA was synthesized from 1 μg of the cRNA, and biotin-labeled cRNA was obtained from the resulting double-stranded cDNA by using Ampliscribe transcription kit (Epicentre Technologies, Madison, MI) and biotin-CTP (Life Technologies, Rockville, Md.) for the substrate. Next, the labeled cRNA was hybridized with 3 types of DNA microarrays (HO-1, HO-2, and HO-3; Mergen, San Leandro, Calif.) each having 1152 types of oligonucleotides from human gene spotted thereon. This means that expression levels for 3456 types of genes in total were analyzed for each of the AC133 positive cell from each donor. The DNA microarray after the hybridization was brought in contact with streptavidin, and the microarray was reacted with anti-streptavidin primary antibody, and then, with Cy3 conjugated secondary antibody. It is to be noted that the streptavidin, the anti-streptavidin primary antibody, and the Cy3-conjugated secondary antibody were those purchased from Mergen (San Leandro, Calif.), and the experiment was carried out in accordance with the protocol attached to the ExpressChip DNA microarray system (Mergen, San Leandro, Calif.).

Next, fluorescence intensity of each gene spot was measured with GMS418 array scanner (Affymetrix, Santa Clara, Calif.), and the measurements were converted into a value by using ImaGene 3.0 (BioDiscovery, Los Angeles, Calif.). The resulting data were statistically processed by GeneSpring 3.2 (Silicon Genetics, Redwood, Calif.). To be more specific, signal intensity of all spots were digitized in every hybridization, and the values were normalized for the average spot intensity. The resulting data were subjected to cluster analysis to obtain a genetic tree (also referred to as a dendrogram) wherein genes having similar expression profile are arranged near a single tree. By this procedure, gene clusters which are active only in a particular disease phase or in a subgroup of the disease phase were found. In addition, in order to visualize the phase-specific genes in the chronic myeloid leukemia, average expression intensity of the genes was calculated for the chronic phase, the accelerated phase, and the blast crisis phase, and the genetic tree was again depicted on the bases of such values. The genes exhibiting expression level alteration in a manner specific to the chronic phase, the accelerated phase and the blast crisis phase were picked out from this average tree. The genes selected are summarized in Table 2. The unit of the expressed amount shown in Table 2 is arbitrary, and the values of 0.6 or less means that the expression was below the detection limit. The values are not absolute, and may vary by the conditions of the patient, preparation of the mRNA, and the conditions of the DNA microarray.

TABLE 2
Genes which showed alteration in the expression level

| Amount | | | | |
|---|---|---|---|---|
| expressed | | | Accelerated | Blast crisis |
| (Arbitrary unit) | Normal | Chronic phase | phase | phase |
| IFIT-2 | 17.8 | 128.7 | 7.9 | 3.8 |
| LAGE-1 | <0.6 | 20.1 | <0.6 | <0.6 |
| BAGE | <0.6 | 19.0 | <0.6 | <0.6 |
| DDB1 | 1.0 | 4.5 | 17.3 | 23.9 |
| ETS-2 | 1.5 | 2.0 | 4.7 | 7.5 |
| PIASy | 26.1 | 52.4 | 4.2 | 10.0 |
| PIASx-α, β | 5.8 | 16.8 | 1.7 | 1.5 |
| DAPK3 | 63.7 | 109.4 | 9.2 | 20.7 |

39 genes were found to exhibit specific expression in the chronic phase. Among such chronic phase-specific genes, the most notable was interferon-induced protein with tetratricopeptide repeats 2 (IFIT-2) gene (SEQ ID NO: 1). Transcription of the IFIT-2 gene was very active in the 5 samples (CP-3 to CP-7) out of the 7 samples taken from chronic phase donors (CP-1 to CP-7), while very weak or no transcription was found in the samples taken from normal donors or chronic myeloid leukemia patients of other disease phase. In samples CP-6 and CP-7, LAGE-1 gene (SEQ-ID NO: 3) and B melanoma antigen (BAGE) gene (SEQ ID NO: 5) were also found to exhibit specific expression.

By using the same average tree, attempt was made to identify genes whose expression is not found in the AC133 positive cells from a normal donor or a patient of chronic phase while the expression is induced in the AC133 positive cell from the patient of accelerated phase or blast crisis phase. As a result, DNA damage binding protein (DDB1) gene (SEQ ID NO: 7) and ETS2 transcription factor gene (SEQ ID NO: 9) were found. DDB1 is involved in the DNA repair mechanism, and loss of its activity is related to complementation group E of xeroderma pigmentosum. Loss of stability of the DNA and various types of chromosome aberration are often found in the blast from chronic myeloid leukemia patient of advanced phase. Accordingly, the induction of the DDB1 gene expression as seen here is believed to reflect the DNA damage accumulated in the leukemia cell of the patients in the accelated phase or the blast crisis phase. In addition, both DDB1 gene and ETS2 gene are induced in CP-1 patient, indicating the possibility that this patient is in the course of the transfer to the accelerated phase or the blast crisis phase.

By using the same average tree, attempt was also made to identify genes whose expression is reduced with the phase transfer to the accelerated phase or the blast crisis phase. 21 genes were then found to exhibit high expression in the AC133 positive cells taken from normal donors or patients in their chronic phase while the expression is reduced in the AC133 positive cells taken from patients in their accelerated phase or blast crisis phase. Among such genes, PIASy gene (SEQ ID NO: 11), PIASx-α gene (SEQ ID NO: 13), and PIASx-β gene (SEQ ID NO: 15) exhibited most remarkable reduction. PIAS 1 and PIAS3 bind to STAT1 and STAT3, respectively, and suppress their activity. Accodingly, although little is known for a protein to which PIASy binds, it is postulated that PIASy may have some function to regulate a member of STAT pathway proteins. It is known that STAT protein is frequently phosphorylated in excess and is activated in the cells derived from individuals suffering from various types of leukemia. Activation of the STAT protein occurs upon introduction of the BCR-ABL kinase into the hematopoietic system, and this indicates the possibility of the STAT protein being a mediator of the BCR-ABL signaling. It was thus speculated that overexpression of PIASy hinders transport of the activated STAT proteins in CML blast cells in chronic phase, while loss of PIASy results in uncontrolled blast cell growth that leads to the progress of the disease phase.

### EXAMPLE 4

### Confirmation of Expression Level by Real Time PCR

For the IFIT-2 gene and the PIASy gene which had been identified to be the phase-specific genes in chronic myeloid leukemia in Example 3, real time PCR was carried out to confirm their expression level in the AC133 positive cells from normal donors and patients of each disease phases. First, cDNA was prepared from the AC133 positive cells of patients by the procedure as described in Examples 1 and 2. The resulting cDNA was subjected to PCR using SYBR Green PCR Core Reagents (PE Applied Biosystems, Foster City, Calif.). In the PCR, the primers used for the amplification IFIT-2 gene were 5′-ctgcggtatggcaactttcag-3′ (SEQ ID NO: 20) and 5′-aggaatgccaagacatgcaa-3′ (SEQ ID NO: 21), and the primers used for the amplification of PIASy gene were 5′-aactacggcaagagctactcggtg-3′ (SEQ ID NO: 22) and 5′-gttcatctgcaggtagaagacggc-3′ (SEQ ID NO: 23). β-actin gene was also used for the control according to the method used in the art. ABI PRISM 7700 Sequence Detection System (PE Applied Biosystems) was used to determine the cycle threshold (CT value) at which the exponential growth of the PCR product is first detected in the real time monitoring of the incorporation of the SYBR Green dye to the PCR product. Amount of the target gene in relation to the β-actin mRNA amount was calculated from the CT value of the cDNA corresponding to the β-actin gene and the target gene. The amount of IFIT-2 gene and PIASy gene expressed in the AC133 positive cells from the patients of each disease phase was then found to be consistent with the measurement of Example 3 obtained by using a DNA microarray confirming the reproducibility in the expression of these genes.

### EXAMPLE 5

### Effects of PIASy Gene Introduction

PIASy gene which had been identified in Example 3 to be a phase-specific gene for the chronic myeloid leukemia was introduced in a cell from a chronic myeloid leukemia patient of blast crisis phase to thereby examine the effects of the PIASy gene expression.

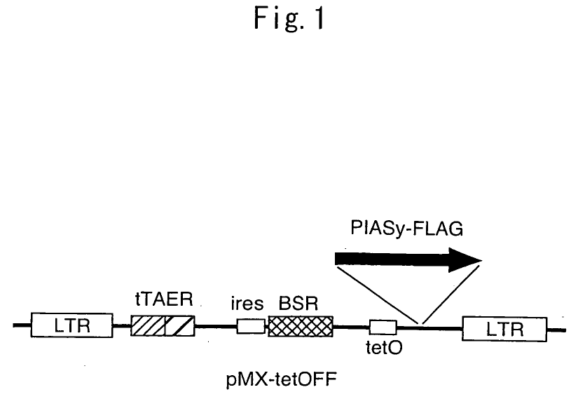

The expression vector containing the PIASy gene was constructed as described below. First, pMX-tetOFF vector was prepared by introducing the cDNA coding for tTAER and ires-BSR and tetO fragments in pMX retroviral vector (Onishi et al.: Exp. Hematol., 24,: 324-329 (1996)). Next, PIASy-F gene coding for PIASy (PIASy-F) having FLAG epitope tag (Eastman Kodak) conjugated at the C terminal was amplified by the PCR using PIASy cDNA (Helix Research Institute) for the template. PIASy-F gene was then ligated to the downstream of the tetO fragment in the pMX-tetOFF vector to thereby construct the expression vector (pMX-tetOFF/PIASy-F) (FIG. 1). The resulting expression vector was introduced in a packaging cell line (PA317, American Type Culture Collection).

In this Example, KCL22 cells (American Type Culture Collection) were used for the cell from a chronic myeloid leukemia patient in the blast crisis phase. KCL22 cells were infected with the pMX-tetOFF/PIASy-F in the culture supernatant of the PA317 by treating the cell in RPMI1640 medium supplemented with 10% fetal bovine serum (FBS) for 24 hours in the presence RetroNectin (Takara Shuzo). The KCL22 cells recovered were then cultivated in the RPMI1640 medium supplemented with 10% fetal bovine serum (FBS) further supplemented with 5 μg/ml of blasticidin and 1 μg/ml of tetracycline. PIASy-F gene in the expression vector was then expressed by cultivating the KCL22 cells in the RPMI1640 medium supplemented with 10% fetal bovine serum (FBS) having 2 μM of 17β-estradiol added thereto.

It is to be noted that the expression vector (pMX-tetOFF/PIASy-F) shown in FIG. 1 does not express the gene (PIASy-F gene) in the downstream of tetO when tetracycline is present and β estradiol is absent. In contrast, the expression vector (pMX-tetOFF/PIASy-F) expresses the gene (PIASy-F gene) in the downstream of tetO when tetracycline is absent and β estradiol is present. The effects of the PIASy gene expression was confirmed by examining the growth of KCL22 cells carrying pMX-tetOFF/PIASy-F introduced therein.

To be more specific, the KCL22 cells carrying pMX-tetOFF introduced therein and the KCL22 cells carrying pMX-tetOFF/PIASy-F introduced therein were evaluated for their cell number and the survival rate both under the conditions that allow expression of the gene in the downstream of tetO and under the conditions that do not allow expression of the gene in the downstream of tetO. It is to be noted that KCL22 cells cultivated in RPMI1640 medium supplemented with 10% fetal bovine serum (FBS) in both conditions.

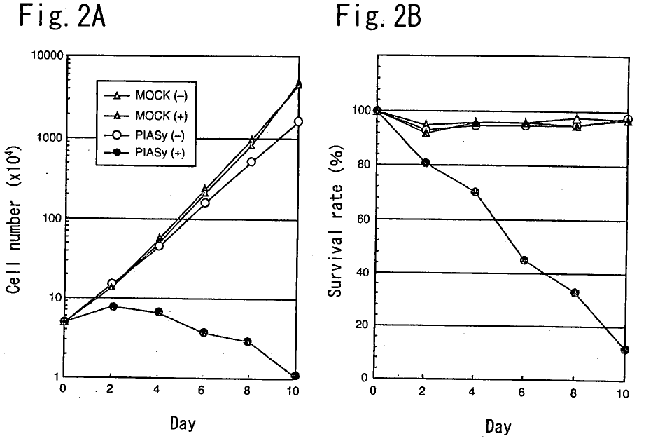

The results of evaluating the cell number and the survival rate are shown in FIGS. 2A and 2B, respectively. As shown in FIGS. 2A and 2B, both the cell number and the survival rate decreased in proportion to the growing period of the KCL22 cells expressing PIASy gene. On the contrary, the cell number and the survival rate for the KCL22 cells carrying pMX-tetOFF/PIASy-F, which show no expression of the PIASy gene (“PIASy (−)” in FIG. 2), were not different from those for the KCL22 cells carrying pMX-tetOFF irrespective of the growing period of the cells.

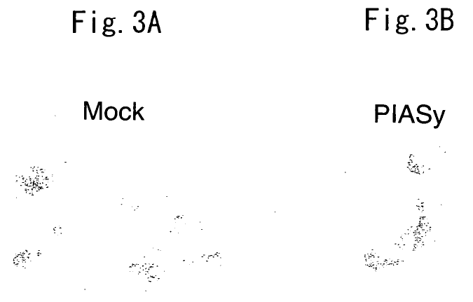

FIGS. 3A and 3B are photomicrographs (×400) of the KCL22 cells carrying pMX-tetOFF introduced therein and the KCL22 cells carrying pMX-tetOFF/PIASy-F introduced therein when these cells were cultivated for 6 days under the conditions that allow expression of the gene in the downstream of tetO. FIG. 3A is the photomicrograph of the KCL22 cells carrying pMX-tetOFF introduced therein (MOCK), and FIG. 3B is the photomicrograph of the KCL22 cells carrying pMX-tetOFF/PIASy-F introduced therein (PIASy). It is to be noted that both KCL22 cells were stained with Wright-Giemsa solution. As shown in FIGS. 3A and 3B, the KCL22 cells carrying pMX-tetOFF introduced therein showed normal phenotype (intermediate sized cell having a basoplilic cytoplasm, high proportion of nucleus in relation to cytoplasm, and good nuclear structure) while the KCL22 cells carrying pMX-tetOFF/PIASy-F introduced therein (PIASy) showed apoptotic change (size reduction of the cell, and contraction and fragmentation of the nucleus).

FIGS. 2 and 3 revealed that expression of the PIASy-F gene in the KCL22 cells carrying pMX-tetOFF/PIASy-F introduced therein results in the induction of apoptosis in the KCL22 cells. PIASy protein has been recently demonstrated to inhibit activity of STAT1, and it is postulated that the apoptosis as described above is induced by the inhibition of the STAT1 in the KCL22 cells. High-level expression of the PIASy gene has been observed in the AC133 positive cells derived from each of normal donors and chronic patients, and it may relate to the reduced risk of chronic patients to progress into the blast crisis phase.

### INDUSTRIAL APPLICABILITY

Provided by this invention are a method for detecting chronic myeloid leukemia; a method for evaluating a compound for its effectiveness in treating or preventing chronic myeloid leukemia; a compound which is effective in treating or preventing chronic myeloid leukemia containing the compound obtained by such evaluation method; a therapeutic or prophylactic agent for chronic myeloid leukemia containing such compound; and a method for determining the risk of being susceptible for chronic myeloid leukemia.

1. A method for detecting chronic myeloid leukemia comprising the step of measuring expression level of a gene selected from the group consisting of IFIT-2 gene, LAGE-I gene, BAGE gene, DDB1 gene, ETS2 gene, PIASy gene, PIASx-α gene, PIASx-β gene, and DAPK3 gene in a test tissue or a test cell; or determining expression profile of a gene group containing at least one of said genes so as to thereby detect chronic myeloid leukemia.

2. A method for detecting chronic myeloid leukemia according to claim 1 wherein the test cell is a bone marrow cell or a blood cell.

3. A method for detecting chronic myeloid leukemia according to claim 2 wherein the bone marrow cell or the blood cell is pluripotent hematopoietic stem cell.

4. A method for detecting chronic myeloid leukemia according to claim 3 wherein the pluripotent hematopoietic stem cell is a AC 133 protein-positive cell.

5. A method for detecting chronic myeloid leukemia according to claim 1 wherein the measurement of the gene expression level or the determination of the expression profile of the gene group is carried out at the level of mRNA.

6. A method for detecting chronic myeloid leukemia according to claim 5 wherein the measurement of the gene expression level or the determination of the expression profile of the gene group at the level of mRNA is carried out by means of gene amplification and/or by using a DNA microarray.

7. A method for detecting chronic myeloid leukemia according to claim 1 wherein the measurement of the gene expression level or the determination of the expression profile of the gene group is carried out at the level of protein.

8. A method for detecting chronic myeloid leukemia according to claim 7 wherein the measurement of the gene expression level or the determination of the expression profile of the gene group at the level of protein is carried out by means of immunological method and/or by using a protein chip.

9. A method for determining phase of chronic myeloid leukemia wherein the phase of the chronic myeloid leukemia is determined by using the detection method of chronic myeloid leukemia according to any one of claims 1 to 8.

10. A method for determining phase of chronic myeloid leukemia according to claim 9 wherein the phase of the chronic myeloid leukemia is selected from the group consisting of chronic phase, accelerated phase, and blastic phase.

11. A method for evaluating a compound for its effectiveness in treating or preventing chronic myeloid leukemia, comprising the steps of:
(a) administering the compound to be evaluated to a test animal or a test cell, or bringing the compound in contact with a test animal or a test cell; and
(b) confirming whether the compound regulates expression level of a gene selected from the group consisting of IFIT-2 gene, LAGE-1 gene, BAGE gene, DDB1 gene, ETS2 gene, PIASy gene, PIASx-α gene, PIASx-β gene, and DAPK3 gene, or a gene which is functionally equivalent to such gene in test animal or the test cell.

12. A method for evaluating a compound for its effectiveness in treating or preventing chronic myeloid leukemia, comprising the steps of:
(a) bringing the compound to be evaluated in contact with a test animal or a test cell containing a fusion gene of an expression-regulatory region for a gene selected from the group consisting of IFIT-2 gene, LAGE-1 gene, BAGE gene, DDB1 gene, ETS2 gene, PIASy gene, PIASx-α gene, PIASx-β gene, and DAPK3 gene; and a reporter gene; and
(b) measuring the level of the reporter gene expressed in the test animal or the test cell to evaluate the compound.

13. A method for evaluating a compound for its effectiveness in treating or preventing chronic myeloid leukemia, comprising the steps of:
(a) bringing the compound to be evaluated in contact with a protein encoded by a gene selected from the group consisting of IFIT-2 gene, LAGE-1 gene, BAGE gene, DDB1 gene, ETS2 gene, PIASy gene, PIASx-α gene, PIASx-β gene, and DAPK3 gene; and
(b) measuring the activity of the protein to evaluate the compound.

14. A method for evaluating a compound for its effectiveness in treating or preventing chronic myeloid leukemia, comprising the steps of:
(a) bringing the compound to be evaluated in contact with a STAT protein whose activity is inhibited by a protein encoded by PIASy gene, PIASx-α gene, or PIASx-β gene; and
(b) measuring the activity of the STAT protein to evaluate the compound.

15. A therapeutic or prophylactic agent for chronic myeloid leukemia comprising a compound obtained by the evaluation method of any one of claims 11 to 14 as an active ingredient.

16. A reagent for research purpose, or an agent for detection, treatment, or prevention of chronic myeloid leukemia comprising an antibody directed against a protein encoded by a gene selected from the group consisting of IFIT-2 gene, LAGE-1 gene, BAGE gene, DDB1 gene, ETS2 gene, PIASy gene, PIASx-α gene, PIASx-β gene, and DAPK3 gene.

17. A method for evaluating the risk of being susceptible for chronic myeloid leukemia comprising the step of detecting a genetic polymorphism which causes abnormality in expression level of a gene selected from the group consisting of IFIT-2 gene, LAGE-1 gene, BAGE gene, DDB1 gene, ETS2 gene, PIASy gene, PIASx-α gene, PIASx-β gene, and DAPK3.

18. A method for evaluating the risk of being susceptible to chronic myeloid leukemia comprising the step of detecting a genetic polymorphism which causes abnormality in biological activity of the protein encoded by a gene selected from the group consisting of IFIT-2 gene, LAGE-1 gene, BAGE gene, DDB1 gene, ETS2 gene, PIASy gene, PIASx-α gene, PIASx-β gene, and DAPK3 gene.

19. A therapeutic or prophylactic agent for chronic myeloid leukemia comprising an antisense nucleic acid which is capable of specifically hybridizing with mRNA of a gene selected from the group consisting of IFIT-2 gene, LAGE-1 gene, BAGE gene, DDB1 gene, and ETS2 gene.

20. A therapeutic or prophylactic agent for chronic myeloid leukemia comprising a ribozyme capable of specifically cleaving mRNA of a gene selected from the group consisting of IFIT-2 gene, LAGE-1 gene, BAGE gene, DDB1 gene, and ETS2 gene.


SEQUENCE LISTING (23 sequences)

SEQ ID NO: 1 - DNA, 2581 bp, Homo sapiens
  CDS (76)..(1494)
agaagaggaagatttctgaagagtgcagctgcctgaaccgagccctgccgaacagctgagaattgcagtgcaaccatgagtgagaacaataagaattccttggagagcagcctacggcaactaaaatgccatttcacctggaacttgatggagggagaaaactccttggatgattttgaagacaaagtattttaccggactgagtttcagaatcgtgaattcaaagccacaatgtgcaacctactggcctatctaaagcacctcaaagggcaaaacgaggcagccctggaatgcttacgtaaagctgaagagttaatccagcaagagcatgctgaccaggcagaaatcagaagtctggtcacctggggaaactatgcctgggtctactatcacatgggccgactctcagacgttcagatttatgtagacaaggtgaaacatgtctgtgagaagttttccagtccctatagaattgagagtccagagcttgactgtgaggaagggtggacacggttaaagtgtggaggaaaccaaaatgaaagagcgaaggtgtgctttgagaaggctctggaaaagaagccaaagaacccagaattcacctctggactggcaatagcaagctaccgtctggacaactggccaccatctcagaacgccattgaccctctgaggcaagccattcggctgaatcctgacaaccagtaccttaaagtcctcctggctctgaagcttcataagatgcgtgaagaaggtgaagaggaaggtgaaggagagaagttagttgaagaagccttggagaaagccccaggtgtaacagatgttcttcgcagtgcagccaagttttatcgaagaaaagatgagccagacaaagcgattgaactgcttaaaaaggctttagaatacataccaaacaatgcctacctgcattgccaaattgggtgctgctatagggcaaaagtcttccaagtaatgaatctaagagagaatggaatgtatgggaaaagaaagttactggaactaataggacacgctgtggctcatctgaagaaagctgatgaggccaatgataatctcttccgtgtctgttccattcttgccagcctccatgctctagcagatcagtatgaagacgcagagtattacttccaaaaggaattcagtaaagagcttactcctgtagcgaaacaactgctccatctgcggtatggcaactttcagctgtaccaaatgaagtgtgaagacaaggccatccaccactttatagagggtgtaaaaataaaccagaaatcaagggagaaagaaaagatgaaagacaaactgcaaaaaattgccaaaatgcgactttctaaaaatggagcagattctgaggctttgcatgtcttggcattccttcaggagctgaatgaaaaaatgcaacaagcagatgaagactctgagaggggtttggagtctggaagcctcatcccttcagcatcaagctggaatggggaatgaagaatagagatgtggtgcccactaggctactgctgaaagggagctgaaattcctccaccaagttggtattcaaaatatgtaatgactggtatggcaaaagattggactaagacactggccataccactggacagggttatgttaacacctgaattgctgggtcttgagagagcccaaggagttctgggagagggaccagattggggggtaggtccacgggcttggtgatagaattatttctcgattgacttcttgagtgccaatttgaactgtaaccatttgcttagtcacctttagtggagtaatcctactgggcttgtttctatatttatataaagcagccaaatccttcatgtaatattgaagtccatttttgcaatgttgttccatacttggagtcattttgcagcccatagaggttagtcctgcatagccagtaatgtgctaagttcatccaaaagctggcggaccaaagtctaaatagggctcagtatccccctatcgcttatctctgcctccttcctcctccttcccagtctatcatcaaccttgagtattctacacatgtgaattcaagtgcctgattaattgaggtggcaacatagtttgagacgagggcagagaacaggaagatacatagctagaagcgacgggtacaaaaagcaatgtgtacaagaagactttcagcaagtatacagagagttcaccctcctcatagtcataatgtagcaagtaaagaatgagaattgggttcttctacaatacaactagaaaccaacaattaatggtatttctttaaaacctgtgtgaaaaaataaatgttgtccaccagtagggataggggaaaagtaaccaaaagagagaaagagaaaggaatgctggtttatctttgtagattgtaatcgaatggagaaatttgcagtattttagccactattaggaattttttttttttgtaaaatgaagactgaactctgttcaaatgctttcatgaacctggtttgagacggtaggaaagcaacaaaacgtgggaacctggtgactaagggcctggtgcaaggacttgggaaatgtcattgataataaa

SEQ ID NO: 2 - protein, 472 aa, Homo sapiens
MSENNKNSLESSLRQLKCHFTWNLMEGENSLDDFEDKVFYRTEFQNREFKATMCNLLAYLKHLKGQNEAALECLRKAEELIQQEHADQAEIRSLVTWGNYAWVYYHMGRLSDVQIYVDKVKHVCEKFSSPYRIESPELDCEEGWTRLKCGGNQNERAKVCFEKALEKKPKNPEFTSGLAIASYRLDNWPPSQNAIDPLRQAIRLNPDNQYLKVLLALKLHKMREEGEEEGEGEKLVEEALEKAPGVTDVLRSAAKFYRRKDEPDKAIELLKKALEYIPNNAYLHCQIGCCYRAKVFQVMNLRENGMYGKRKLLELIGHAVAHLKKADEANDNLFRVCSILASLHALADQYEDAEYYFQKEFSKELTPVAKQLLHLRYGNFQLYQMKCEDKAIHHFIEGVKINQKSREKEKMKDKLQKIAKMRLSKNGADSEALHVLAFLQELNEKMQQADEDSERGLESGSLIPSASSWNGE

SEQ ID NO: 3 - DNA, 755 bp, Homo sapiens
  CDS (53)..(595)
tcctcgtgggccctgaccttctctctgagagccgggcagaggctccggagccatgcaggccgaaggccagggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgagaggaggcgccccgcggggtccgcatggcggtgccgcttctgcgcaggatggaaggtgcccctgcggggccaggaggccggacagccgcctgcttcagttgcacatcacgatgcctttctcgtcgcccatggaagcggagctggtccgcaggatcctgtcccgggatgccgcacctctcccccgaccaggggcggttctgaaggacttcaccgtgtccggcaacctactgtttatccgactgactgctgcagaccaccgccaactgcagctctccatcagctcctgtctccagcagctttccctgttgatgtggatcacgcagtgctttctgcccgtgtttttggctcaggctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaagctgagctacgaaaaaaaaa

SEQ ID NO: 4 - protein, 180 aa, Homo sapiens
MQAEGQGTGGSTGDADGPGGPGIPDGPGGNAGGPGEAGATGGRGPRGAGAARASGPRGGAPRGPHGGAASAQDGRCPCGARRPDSRLLQLHITMPFSSPMEAELVRRILSRDAAPLPRPGAVLKDFTVSGNLLFIRLTAADHRQLQLSISSCLQQLSLLMWITQCFLPVFLAQAPSGQRR

SEQ ID NO: 5 - DNA, 1004 bp, Homo sapiens
  CDS (201)..(332)
cgccaatttagggtctccggtatctcccgctgagctgctctgttcccggcttagaggaccaggagaagggggagctggaggctggagcctgtaacaccgtggctcgtctcactctggatggtggtggcaacagagatggcagcgcagctggagtgttaggagggcggcctgagcggtaggagtggggctggagcagtaagatggcggccagagcggtttttctggcattgtctgcccagctgctccaagccaggctgatgaaggaggagtcccctgtggtgagctggaggttggagcctgaagacggcacagctctgtgcttcatcttctgaggttgtggcagccacggtgatggagacggcagctcaacaggagcaataggaggagatggagtttcactgtgtcagccaggatggtctcgatctcctgacctcgtgatccgcccgccttggccttccaaagtgccgagattacagcgatgtgcattttgtaagcactttggagccactatcaaatgctgtgaagagaaatgtacccagatgtatcattatccttgtgctgcaggagccggctcctttcaggatttcagtcacatcttcctgctttgtccagaacacattgaccaagctcctgaaagatgtaagtttactacgcatagacttttaaacttcaaccaatgtatttactgaaaataacaaatgttgtaaattccctgagtgttattctacttgtattaaaaggtaataatacataatcattaaaatctgagggatcattgccagagattgttggggagggaaatgttatcaacggtttcattgaaattaaatccaaaaagttatttcctcagaaaaatcaaataaagtttgcatgttttttattcttaaaacattttaaaaaccactgtagaatgatgtaaatagggactgtgcagtatttctgacatatactataaaattattaaaaagtcaatcagtattcaacatcttttacactaaaaagcc

SEQ ID NO: 6 - protein, 43 aa, Homo sapiens
MAARAVFLALSAQLLQARLMKEESPVVSWRLEPEDGTALCFIF

SEQ ID NO: 7 - DNA, 4193 bp, Homo sapiens
  CDS (101)..(3523)
gtggagttcgctgcggctgttgggggccacctgtcttttcgcttgtgcccctctttctagtgtcgcgctcgagtcccgacgggccgctccaagcctcgacatgtcgtacaactacgtggtaacggcccagaagcccaccgccgtgaacggctgcgtgaccggacactttacttcggccgaagacttaaacctgttgattgccaaaaacacgagattagagatctatgtggtcaccgccgaggggcttcggcccgtcaaagaggtgggcatgtatgggaagattgcggtcatggagcttttcaggcccaagggggagagcaaggacctgctgtttatcttgacagcgaagtacaatgcctgcatcctggagtataaacagagtggcgagagcattgacatcattacgcgagcccatggcaatgtccaggaccgcattggccgcccctcagagaccggcattattggcatcattgaccctgagtgccggatgattggcctgcgtctctatgatggccttttcaaggttattccactagatcgcgataataaagaactcaaggccttcaacatccgcctggaggagctgcatgtcattgatgtcaagttcctatatggttgccaagcacctactatttgctttgtctaccaggaccctcaggggcggcacgtaaaaacctatgaggtgtctctccgagaaaaggaattcaataagggcccttggaaacaggaaaatgtcgaagctgaagcttccatggtgatcgcagtcccagagccctttgggggggccatcatcattggacaggagtcaatcacctatcacaatggtgacaaatacctggctattgcccctcctatcatcaagcaaagcacgattgtgtgccacaatcgagtggaccctaatggctcaagatacctgctgggagacatggaaggccggctcttcatgctgcttttggagaaggaggaacagatggatggcaccgtcactctcaaggatctccgtgtagaactccttggagagacctctattgctgagtgcttgacataccttgataatggtgttgtgtttgtcgggtctcgcctgggtgactcccagcttgtgaagctcaacgttgacagtaatgaacaaggctcctatgtagtggccatggaaacctttaccaacttaggacccattgtcgatatgtgcgtggtggacctggagaggcaggggcaggggcagctggtcacttgctctggggctttcaaggaaggttctttgcggatcatccggaatggaattggaatccacgagcatgccagcattgacttaccaggcatcaaaggattatggccactgcggtctgaccctaatcgtgagactgatgacactttggtgctctcttttgtgggccagacaagagttctcatgttaaatggagaggaggtagaagaaaccgaactgatgggtttcgtggatgatcagcagactttcttctgtggcaacgtggctcatcagcagcttatccagatcacttcagcatcggtgaggttggtctctcaagaacccaaagctctggtcagtgaatggaaggagcctcaggccaagaacatcagtgtggcctcctgcaatagcagccaggtggtggtggctgtaggcagggccctctactatctgcagatccatcctcaggagctccggcagatcagccacacagagatggaacatgaagtggcttgcttggacatcaccccattaggagacagcaatggactgtcccctctttgtgccattggcctctggacggacatctcggctcgtatcttgaagttgccctcttttgaactactgcacaaggagatgctgggtggagagatcattcctcgctccatcctgatgaccacctttgagagtagccattacctcctttgtgccttgggagatggagcgcttttctactttgggctcaacattgagacaggtctgttgagcgaccgtaagaaggtgactttgggcacccagcccaccgtattgaggacttttcgttctctttctaccaccaacgtctttgcttgttctgaccgccccactgtcatctatagcagcaaccacaaattggtcttctcaaatgtcaacctcaaggaagtgaactacatgtgtcccctcaattcagatggctatcctgacagcctggcgctggccaacaatagcaccctcaccattggcaccatcgatgagatccagaagctgcacattcgcacagttcccctctatgagtctccaaggaagatctgctaccaggaagtgtcccagtgtttcggggtcctctccagccgcattgaagtccaagacacgagtgggggcacgacagccttgaggcccagcgctagcacccaggctctgtccagcagtgtaagctccagcaagctgttctccagcagcactgctcctcatgagacctcctttggagaagaggtggaggtgcacaacctacttatcattgaccaacacacctttgaagtgcttcatgcccaccagtttctgcagaatgaatatgccctcagtctggtttcctgcaagctgggcaaagaccccaacacttacttcattgtgggcacagcaatggtgtatcctgaagaggcagagcccaagcagggtcgcattgtggtctttcagtattcggatggaaaactacagactgtggctgaaaaggaagtgaaaggggccgtgtactctatggtggaatttaacgggaagctgttagccagcatcaatagcacggtgcggctctatgagtggacaacagagaaggagctgcgcactgagtgcaaccactacaacaacatcatggccctctacctgaagaccaagggcgacttcatcctggtgggcgaccttatgcgctcagtgctgctgcttgcctacaagcccatggaaggaaactttgaagagattgctcgagactttaatcccaactggatgagtgctgtggaaatcttggatgatgacaattttctgggggctgaaaatgcctttaacttgtttgtgtgtcaaaaggatagcgctgccaccactgacgaggagcggcagcacctccaggaggttggtcttttccacctgggcgagtttgtcaatgtcttttgccacggctctctggtaatgcagaatctgggtgagacttccacccccacacaaggctcggtgctcttcggcacggtcaacggcatgatagggctggtgacctcactgtcagagagctggtacaacctcctgctggacatgcagaatcgactcaataaagtcatcaaaagtgtggggaagatcgagcactccttctggagatcctttcacaccgagcggaagacagaaccagccacaggtttcatcgacggtgacttgattgagagtttcctggatattagccgccccaagatgcaggaggtggtggcaaacctacagtatgacgatggcagcggtatgaagcgagaggccactgcagacgacctcatcaaggttgtggaggagctaactcggatccattagccaagggcagggggccccctttctgaccctccccaaaggctttgccctgctgccctccccctcctctccaccatcgtcttcttggccatgggaggcctttccctaagccagctgcccccagagccacagttcccctatgtggaagtggggcgggcttcatagagacttgggaatgagctgaaggtgaaacattttctccctggatttttaccagtctcacatgattccagccatcaccttagaccaccaagccttgattggtgttgccagttgtcctccttccggggaaggattttgcagttctttggctgaaaggaagctgtgcgtgtgtgtgtgtgtatgtgtgtgtgtgtatgtgtatctcacactcatgcaatgtcctctttttatttagattggcagtgtagggagttgtgggtagtggggaagagggttaggagggtttcattgtctgtgaagtgagaccttccttttacttttcttctattgcctctgagagcatcagcctagaggcctgactgccaagccatgggtagcctgggtgtaaaacctggagatggtggatgatccccacgccacagcccttttgtctctgcaaactgccttcttcggaaagaagaaggtgggaggatgtgaattgttagtttctgagttttaccaaataaagtag

SEQ ID NO: 8 - protein, 1140 aa, Homo sapiens [sequence omitted]

SEQ ID NO: 9 - DNA, 2269 bp, Homo sapiens
  CDS (292)..(1701)
ccgtttcctcccctcccctccactcggccgtccctccttcctcctccctcctccctcctcctcccgctcctgaagagcgcgccgcgtgggggacggcccggttacttcctccagagactgacgagtgcggtgtcgctccagctcagagctcccggagccgcccggccagcgtccggcctccctgatcgtctctggccggcgccctcgccctcgcccggcgcgcaccgagcagccgcgggcgccgagcagccaccgtcccgaccaagcgccggccctgcccgcagcggcaggatgaatgatttcggaatcaagaatatggaccaggtagcccctgtggctaacagttacagagggacactcaagcgccagccagcctttgacacctttgatgggtccctgtttgctgtttttccttctctaaatgaagagcaaacactgcaagaagtgccaacaggcttggattccatttctcatgactccgccaactgtgaattgcctttgttaaccccgtgcagcaaggctgtgatgagtcaagccttaaaagctaccttcagtggcttcaaaaaggaacagcggcgcctgggcattccaaagaacccctggctgtggagtgagcaacaggtatgccagtggcttctctgggccaccaatgagttcagtctggtgaacgtgaatctgcagaggttcggcatgaatggccagatgctgtgtaaccttggcaaggaacgctttctggagctggcacctgactttgtgggtgacattctctgggaacatctggagcaaatgatcaaagaaaaccaagaaaagacagaagatcaatatgaagaaaattcacacctcacctccgttcctcattggattaacagcaatacattaggttttggcacagagcaggcgccctatggaatgcagacacagaattaccccaaaggcggcctcctggacagcatgtgtccggcctccacacccagcgtactcagctctgagcaggagtttcagatgttccccaagtctcggctcagctccgtcagcgtcacctactgctctgtcagtcaggacttcccaggcagcaacttgaatttgctcaccaacaattctgggactcccaaagaccacgactcccctgagaacggtgcggacagcttcgagagctcagactccctcctccagtcctggaacagccagtcgtccttgctggatgtgcaacgggttccttccttcgagagcttcgaagatgactgcagccagtctctctgcctcaataagccaaccatgtctttcaaggattacatccaagagaggagtgacccagtggagcaaggcaaaccagttatacctgcagctgtgctggccggcttcacaggaagtggacctattcagctgtggcagtttctcctggagctgctatcagacaaatcctgccagtcattcatcagctggactggagacggatgggagtttaagctcgccgaccccgatgaggtggcccgccggtggggaaagaggaaaaataagcccaagatgaactacgagaagctgagccggggcttacgctactattacgacaagaacatcatccacaagacgtcggggaagcgctacgtgtaccgcttcgtgtgcgacctccagaacttgctggggttcacgcccgaggaactgcacgccatcctgggcgtccagcccgacacggaggactgaggtcgccgggaccaccctgagccggccccaggctcgtggactgagtgggaagcccatcctgaccagctgcctccgaggacccaggaaaggcaggattgaaaatgtccaggaaagtggccaagaagcagtggccttattgcatcccaaaccacgcctcttgaccaggctgcctcccttgtggcagcaacggcacagctaattctactcacagtgcttttaagtgaaaatggtcgagaaagaggcaccgggaagccgtcctggcgcctggcagtccgtgggacgggatggttctggctgtttgagattctcaaaggagcgagcatgtcgtggacacacacagactatttttagattttcttttgccttttgcaaccaggaacagcaaatgcaaaaactctttgagagggtaggagggtgggaaggaaacaaccatgtcatttcagaagttagtttgtatatattataataatcttataattgttctcagaatcccttaacagttgtatttaacagaaattgtatattgtaatttaaaataattatataactgtatttgaaataagaattc

SEQ ID NO: 10 - protein, 469 aa, Homo sapiens
MNDFGIKNMDQVAPVANSYRGTLKRQPAFDTFDGSLFAVFPSLNEEQTLQEVPTGLDSISHDSANCELPLLTPCSKAVMSQALKATFSGFKKEQRRLGIPKNPWLWSEQQVCQWLLWATNEFSLVNVNLQRFGMNGQMLCNLGKERFLELAPDFVGDILWEHLEQMIKENQEKTEDQYEENSHLTSVPHWINSNTLGFGTEQAPYGMQTQNYPKGGLLDSMCPASTPSVLSSEQEFQMFPKSRLSSVSVTYCSVSQDFPGSNLNLLTNNSGTPKDHDSPENGADSFESSDSLLQSWNSQSSLLDVQRVPSFESFEDDCSQSLCLNKPTMSFKDYIQERSDPVEQGKPVIPAAVLAGFTGSGPIQLWQFLLELLSDKSCQSFISWTGDGWEFKLADPDEVARRWGKRKNKPKMNYEKLSRGLRYYYDKNIIHKTSGKRYVYRFVCDLQNLLGFTPEELHAILGVQPDTED

SEQ ID NO: 11 - DNA, 1533 bp, Homo sapiens
  CDS (1)..(1533)
atggcggcggagctggtggaggccaaaaacatggtgatgagttttcgagtctccgaccttcagatgctcctgggtttcgtgggccggagtaagagtggactgaagcacgagctcgtcaccagggccctccagctggtgcagtttgactgtagccctgagctgttcaagaagatcaaggagctgtacgagacccgctacgccaagaagaactcggagcctgccccacagccgcaccggcccctggaccccctgaccatgcactccacctacgaccgggccggcgctgtgcccaggactccgctggcaggccccaatattgactaccccgtgctctacggaaagtacttaaacggactgggacggttgcccgccaagaccctcaagccagaagtccgcctggtgaagctgccgttctttaatatgctggatgagctgctgaagcccaccgaattagtcccacagaacaacgagaagcttcaggagagcccgtgcatcttcgcattgacgccaagacaggtggagttgatccggaaattccagggaatgcagcccggagttaaagccgtgcaggtcgtcctgagaatctgttactcagacaccagctgccctcaggaggaccagtacccgcccaacatcgctgtgaaggtcaaccacagctactgctccgtcccgggctactacccctccaataagcccggggtggagcccaagaggccgtgccgccccatcaacctcactcacctcatgtacctgtcctcggccaccaaccgcatcactgtcacctgggggaactacggcaagagctactcggtggccctgtacctggtgcggcagctgacctcatcggagctgctgcagaggctgaagaccattggggtaaagcacccggagctgtgcaaggcactggtcaaggagaagctgcgccttgatcctgacagcgagatcgccaccaccggtgtgcgggtgtccctcatctgtccgctggtgaagatgcggctctccgtgccctgccgggcagaaacctgcgcccacctgcagtgcttcgacgccgtcttctacctgcagatgaacgagaagaagcccacctggatgtgccccgtgtgcgacaagccagccccctacgaccagctcatcatcgacgggctcctctcgaagatcctgagcgagtgtgaggacgccgacgagatcgagtacctggtggacggctcgtggtgcccgatccgcgccgaaaaggagcgcagctgcagcccgcagggcgccatcctcgtgctgggcccctcggacgccaatgggctcctgcccgcccccagcgtcaacgggagcggtgccctgggcagcacgggtggcggcggcccggtgggcagcatggagaatgggaagccgggcgccgatgtggtggacctcacgctggacagctcatcgtcctcggaggatgaggaggaggaggaagaggaggaggaagacgaggacgaagaggggccccggcccaagcgccgctgccccttccagaagggcctggtgccggcctgctga

SEQ ID NO: 12 - protein, 510 aa, Homo sapiens
MAAELVEAKNMVMSFRVSDLQMLLGFVGRSKSGLKHELVTRALQLVQFDCSPELFKKIKELYETRYAKKNSEPAPQPHRPLDPLTMHSTYDRAGAVPRTPLAGPNIDYPVLYGKYLNGLGRLPAKTLKPEVRLVKLPFFNMLDELLKPTELVPQNNEKLQESPCIFALTPRQVELIRKFQGMQPGVKAVQVVLRICYSDTSCPQEDQYPPNIAVKVNHSYCSVPGYYPSNKPGVEPKRPCRPINLTHLMYLSSATNRITVTWGNYGKSYSVALYLVRQLTSSELLQRLKTIGVKHPELCKALVKEKLRLDPDSEIATTGVRVSLICPLVKMRLSVPCRAETCAHLQCFDAVFYLQMNEKKPTWMCPVCDKPAPYDQLIIDGLLSKILSECEDADEIEYLVDGSWCPIRAEKERSCSPQGAILVLGPSDANGLLPAPSVNGSGALGSTGGGGPVGSMENGKPGADVVDLTLDSSSSSEDEEEEEEEEEDEDEEGPRPKRRCPFQKGLVPAC

SEQ ID NO: 13 - DNA, 1719 bp, Homo sapiens
  CDS (1)..(1719)
atggcggatttcgaagagttgaggaatatggtttctagttttagggtttctgaactacaagtattactaggctttgctggacggaataaaagtggacgcaagcatgacctcctgatgagggcgctgcatttattgaagagcggctgcagccctgcggttcagattaaaatccgagaattgtatagacgccgatatccacgaactcttgaaggactttctgatttatccacaatcaaatcatcggttttcagtttggatggtggctcatcacctgtagaacctgacttggccgtggctggaatccactcgttgccttccacttcagttacacctcactcaccatcctctcctgttggttctgtgctgcttcaagatactaagcccacatttgagatgcagcagccatctcccccaattcctcctgtccatcctgatgtgcagttaaaaaatctgcccttttatgatgtccttgatgttctcatcaagcccacgagtttagttcaaagcagtattcagcgatttcaagagaagttttttatttttgctttgacacctcaacaagttagagagatatgcatatccagggattttttgccaggtggtaggagagattatacagtccaagttcagttgagactttgcctggcagagacaagttgccctcaagaagataactatccaaatagtctatgtataaaagtaaatgggaagctatttcctttgcctggctatgcaccaccgcctaaaaatgggattgaacagaagcgccctggacgccccttgaatattacatctttagttaggttatcttcagctgtgccaaaccaaatttccatttcttgggcatcagaaattgggaagaattactctatgtctgtatatcttgtacggcagcttacatcagccatgttattacagagattaaaaatgaaaggtattagaaaccctgatcattccagagcactaattaaagaaaaacttactgcagatcctgatagtgaaattgctacaactagccttcgggtatccttgatgtgccctttaggaaaaatgaggctgacaatcccatgccgtgcagtgacttgtacacatctgcagtgttttgatgctgccctctatctacaaatgaatgagaaaaagcccacctggatttgtcctgtgtgtgacaaaaaagctgcctatgaaagtctaatattagatgggctttttatggaaattctcaatgactgttctgatgtagatgagatcaaattccaagaagatggttcttggtgtccaatgagaccgaagaaagaagctatgaaagtatccagccaaccgtgtacaaaaatagaaagttcaagcgtcctcagtaagccttgttcagtgactgtagccagtgaggcaagcaagaagaaagtagatgttattgatcttacaatagaaagctcttctgacgaagaggaagaccctcctgccaaaaggaaatgcatctttatgtcagaaacacaaagcagcccaaccaaaggggttctcatgtatcagccatcttctgtaagggtgcccagtgtgacttcggttgatcctgctgctattccgccttcattaacagactactcagtaccattccaccatacgccaatatcaagcatgtcatcagatttgccaggagaacaaagaagaaatgatattaataatgaactgaagcttggaacatcttctgatactgtgcaacagtga

SEQ ID NO: 14 - protein, 572 aa, Homo sapiens
MADFEELRNMVSSFRVSELQVLLGFAGRNKSGRKHDLLMRALHLLKSGCSPAVQIKIRELYRRRYPRTLEGLSDLSTIKSSVFSLDGGSSPVEPDLAVAGIHSLPSTSVTPHSPSSPVGSVLLQDTKPTFEMQQPSPPIPPVHPDVQLKNLPFYDVLDVLIKPTSLVQSSIQRFQEKFFIFALTPQQVREICISRDFLPGGRRDYTVQVQLRLCLAETSCPQEDNYPNSLCIKVNGKLFPLPGYAPPPKNGIEQKRPGRPLNITSLVRLSSAVPNQISISWASEIGKNYSMSVYLVRQLTSAMLLQRLKMKGIRNPDHSRALIKEKLTADPDSEIATTSLRVSLMCPLGKMRLTIPCRAVTCTHLQCFDAALYLQMNEKKPTWICPVCDKKAAYESLILDGLFMEILNDCSDVDEIKFQEDGSWCPMRPKKEAMKVSSQPCTKIESSSVLSKPCSVTVASEASKKKVDVIDLTIESSSDEEEDPPAKRKCIFMSETQSSPTKGVLMYQPSSVRVPSVTSVDPAAIPPSLTDYSVPFHHTPISSMSSDLPGEQRRNDINNELKLGTSSDTVQQ

SEQ ID NO: 15 - DNA, 1866 bp, Homo sapiens
  CDS (1)..(1866)
atggcggatttcgaagagttgaggaatatggtttctagttttagggtttctgaactacaagtattactaggctttgctggacggaataaaagtggacgcaagcatgacctcctgatgagggcgctgcatttattgaagagcggctgcagccctgcggttcagattaaaatccgagaattgtatagacgccgatatccacgaactcttgaaggactttctgatttatccacaatcaaatcatcggttttcagtttggatggtggctcatcacctgtagaacctgacttggccgtggctggaatccactcgttgccttccacttcagttacacctcactcaccatcctctcctgttggttctgtgctgcttcaagatactaagcccacatttgagatgcagcagccatctcccccaattcctcctgtccatcctgatgtgcagttaaaaaatctgcccttttatgatgtccttgatgttctcatcaagcccacgagtttagttcaaagcagtattcagcgatttcaagagaagttttttatttttgctttgacacctcaacaagttagagagatatgcatatccagggattttttgccaggtggtaggagagattatacagtccaagttcagttgagactttgcctggcagagacaagttgccctcaagaagataactatccaaatagtctatgtataaaagtaaatgggaagctatttcctttgcctggctatgcaccaccgcctaaaaatgggattgaacagaagcgccctggacgccccttgaatattacatctttagttaggttatcttcagctgtgccaaaccaaatttccatttcttgggcatcagaaattgggaagaattactctatgtctgtatatcttgtacggcagcttacatcagccatgttattacagagattaaaaatgaaaggtattagaaaccctgatcattccagagcactaattaaagaaaaacttactgcagatcctgatagtgaaattgctacaactagccttcgggtatccttgatgtgccctttaggaaaaatgaggctgacaatcccatgccgtgcagtgacttgtacacatctgcagtgttttgatgctgccctctatctacaaatgaatgagaaaaagcccacctggatttgtcctgtgtgtgacaaaaaagctgcctatgaaagtctaatattagatgggctttttatggaaattctcaatgactgttctgatgtagatgagatcaaattccaagaagatggttcttggtgtccaatgagaccgaagaaagaagctatgaaagtatccagccaaccgtgtacaaaaatagaaagttcaagcgtcctcagtaagccttgttcagtgactgtagccagtgaggcaagcaagaagaaagtagatgttattgatcttacaatagaaagctcttctgacgaagaggaagaccctcctgccaaaaggaaatgcatctttatgtcagaaacacaaagcagcccaaccaaaggggttctcatgtatcagccatcttctgtaagggtgcccagtgtgacttcggttgatcctgctgctattccgccttcattaacagactactcagtaccattccaccatacgccaatatcaagcatgtcatcagatttgccaggtttggattttctttcccttattccagttgatccccagtactgtcctcctatgtttttggatagtctcacctcacccttaacagcaagcagtacgtctgtcaccaccaccagctcccatgaaagcagtactcatgttagttcatccagcagcaggagtgagacaggggtcataaccagcagtggaagtaacattcctgaaatcatctcattggactaa

SEQ ID NO: 16 - protein, 621 aa, Homo sapiens
MADFEELRNMVSSFRVSELQVLLGFAGRNKSGRKHDLLMRALHLLKSGCSPAVQIKIRELYRRRYPRTLEGLSDLSTIKSSVFSLDGGSSPVEPDLAVAGIHSLPSTSVTPHSPSSPVGSVLLQDTKPTFEMQQPSPPIPPVHPDVQLKNLPFYDVLDVLIKPTSLVQSSIQRFQEKFFIFALTPQQVREICISRDFLPGGRRDYTVQVQLRLCLAETSCPQEDNYPNSLCIKVNGKLFPLPGYAPPPKNGIEQKRPGRPLNITSLVRLSSAVPNQISISWASEIGKNYSMSVYLVRQLTSAMLLQRLKMKGIRNPDHSRALIKEKLTADPDSEIATTSLRVSLMCPLGKMRLTIPCRAVTCTHLQCFDAALYLQMNEKKPTWICPVCDKKAAYESLILDGLFMEILNDCSDVDEIKFQEDGSWCPMRPKKEAMKVSSQPCTKIESSSVLSKPCSVTVASEASKKKVDVIDLTIESSSDEEEDPPAKRKCIFMSETQSSPTKGVLMYQPSSVRVPSVTSVDPAAIPPSLTDYSVPFHHTPISSMSSDLPGLDFLSLIPVDPQYCPPMFLDSLTSPLTASSTSVTTTSSHESSTHVSSSSSRSETGVITSSGSNIPEIISLD

SEQ ID NO: 17 - DNA, 2105 bp, Homo sapiens
  CDS (94)..(1458)
gttgccattaggggactcctgaggtcctatctccaggctgcggtgactgcactttccctggagtggaagctgctggaaggcggaccggccgccatgtccacgttcaggcaggaggacgtggaggaccattatgagatgggggaggagctgggcagcggccagtttgcgatcgtgcggaagtgccggcagaagggcacgggcaaggagtacgcagccaagttcatcaagaagcgccgcctgtcatccagccggcgtggggtgagccgggaggagatcgagcgggaggtgaacatcctgcgggagatccggcaccccaacatcatcaccctgcacgacatcttcgagaacaagacggacgtggtcctcatcctggagctggtctctggcggggagctctttgacttcctggcggagaaagagtcgctgacggaggacgaggccacccagttcctcaagcagatcctggacggcgttcactacctgcactctaagcgcatcgcacactttgacctgaagccggaaaacatcatgctgctggacaagaacgtgcccaacccacgaatcaagctcatcgacttcggcatcgcgcacaagatcgaggcggggaacgagttcaagaacatcttcggcaccccggagtttgtggccccagagattgtgaactatgagccgctgggcctggaggcggacatgtggagcatcggtgtcatcacctatatcctcctgagcggtgcatccccgttcctgggcgagaccaagcaggagacgctcaccaacatctcagccgtgaactacgacttcgacgaggagtacttcagcaacaccagcgagctggccaaggacttcattcgccggctgctcgtcaaagatcccaagcggagaatgaccattgcccagagcctggaacattcctggattaaggcgatccggcggcggaacgtgcgtggtgaggacagcggccgcaagcccgagcggcggcgcctgaagaccacgcgtctgaaggagtacaccatcaagtcgcactccagcttgccgcccaacaacagctacgccgacttcgagcgcttctccaaggtgctggaggaggcggcggccgccgaggagggcctgcgcgagctgcagcgcagccggcggctctgccacgaggacgtggaggcgctggccgccatctacgaggagaaggaggcctggtaccgcgaggagagcgacagcctgggccaggacctgcggaggctacggcaggagctgctcaagaccgaggcgctcaagcggcaggcgcaggaggaggccaagggcgcgctgctggggaccagcggcctcaagcgccgcttcagccgcctggagaaccgctacgaggcgctggccaagcaagtagcctccgagatgcgcttcgtgcaggacctcgtgcgcgccctggagcaggagaagctgcagggcgtggagtgcgggctgcgctaggcgcagtggggtgggccaggccccaggacagccggagctcggcctgcggtgggggcgcttcctgtggacgctgcgcctcccatcgcccgggtgcctgtccttgcccagcgccaccaggctggaggcggagtgggaggagctggagccaggcccgtaagttcgcaggcaggggtgggtgtgggacggggctgcttctctacacagcctctacgctggccttcaccttcacccctgcatcgtcggtgaccctgggaccctccaggcagcgtggcctgtggcaccgtgagggttgggacccaccgaggcgcagaggcggcccgaatgcagccctggttcaggcccggaggagggtttgcgggtagttgcacggacaattcggcggggtgctgcctgttgctgccattagcccaggaggaggtcgtgggacggggagggtgggatggacggcggacaggcagtccccacgctgctgggtggcgccgggcttggtggggtcttccactgtgtgcccttctcgccgaggccggtcccccgggtgtggggtgccctgctgcggactcctccgcgagccccatcgtcgcgcctgtggacgcctaggcaagagcggccctctgcagccaagagaaataaaatactggcttccagat

SEQ ID NO: 18 - protein, 454 aa, Homo sapiens
MSTFRQEDVEDHYEMGEELGSGQFAIVRKCRQKGTGKEYAAKFIKKRRLSSSRRGVSREEIEREVNILREIRHPNIITLHDIFENKTDVVLILELVSGGELFDFLAEKESLTEDEATQFLKQILDGVHYLHSKRIAHFDLKPENIMLLDKNVPNPRIKLIDFGIAHKIEAGNEFKNIFGTPEFVAPEIVNYEPLGLEADMWSIGVITYILLSGASPFLGETKQETLTNISAVNYDFDEEYFSNTSELAKDFIRRLLVKDPKRRMTIAQSLEHSWIKAIRRRNVRGEDSGRKPERRRLKTTRLKEYTIKSHSSLPPNNSYADFERFSKVLEEAAAAEEGLRELQRSRRLCHEDVEALAAIYEEKEAWYREESDSLGQDLRRLRQELLKTEALKRQAQEEAKGALLGTSGLKRRFSRLENRYEALAKQVASEMRFVQDLVRALEQEKLQGVECGLR

SEQ ID NO: 19 - DNA, 66 bp, Artificial Sequence
  synthetic DNA
tctagtcgacggccagtgaattgtaatacgactcactatagggcgttttttttttttttttttttt

SEQ ID NO: 20 - DNA, 21 bp, Artificial Sequence
  synthetic DNA
ctgcggtatggcaactttcag

SEQ ID NO: 21 - DNA, 20 bp, Artificial Sequence
  synthetic DNA
aggaatgccaagacatgcaa

SEQ ID NO: 22 - DNA, 24 bp, Artificial Sequence
  synthetic DNA
aactacggcaagagctactcggtg

SEQ ID NO: 23 - DNA, 24 bp, Artificial Sequence
  synthetic DNA
gttcatctgcaggtagaagacggc